(19) United States
(12) Patent Application Publication
(10) Pub. No.: US 2007/0224253 A1
(43) Pub. Date: Sep. 27, 2007

(54) Transdermal Delivery of Meptazinol

(75) Inventor: Richard Franklin, Hampshire (GB)

Correspondence Address: FROMMER LAWRENCE & HAUG, 745 FIFTH AVENUE- 10TH FL., NEW YORK, NY, 10151, US

(73) Assignee: Shire Pharmaceuticals Inc., Wayne, PA (US)

(21) Appl. No.: 11/614,165

(22) Filed: Dec. 21, 2006

Related U.S. Application Data
(60) Provisional application No. 60/862,114, filed on Oct. 19, 2006.
(60) Provisional application No. 60/753,357, filed on Dec. 21, 2005.

(51) Int. Cl. A61K 9/70; A61K 31/55; A61K 31/7052

(52) U.S. Cl. 424/449; 514/23; 514/212.01

(57) ABSTRACT

A delivery system for the delivery of a salt of meptazinol or meptazinol precursor which increases the bioavailability of meptanizol by an effective amount to provide analgesic relief is disclosed. One embodiment of the delivery system is a transdermal device which increases the skin flux of meptazinol by an effective amount to provide analgesic relief. Also disclosed are methods of providing analgesic relief.

### RELATED APPLICATION AND INCORPORATION BY REFERENCE

U.S. Provisional Application No. 60/862,114, was filed on Oct. 19, 2006 and Ser. No. 60/753,357, was filed on Dec. 21, 2005, both titled “Transdermal Delivery of Meptazinol”.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to the administration of a salt of meptazinol or meptazinol precursor for analgesic purposes and more particularly to a method and device for administering salt of meptazinol or meptazinol precursor to a patient in need thereof over an extended period of time at an essentially constant rate while avoiding first pass metabolism.

### BACKGROUND OF THE INVENTION

Inadequate pain relief continues to represent a major problem for both patients and healthcare professionals. Optimal pharmacologic management of pain requires selection of the appropriate analgesic drug that achieves rapid efficacy with minimal side effects.

Mild analgesics are readily available and over the counter (OTC) analgesics such as paracetamol, are well established in mild pain. Stronger analgesics, as well as often requiring regular medication (3-4 times per day, minimum) have significant side effects e.g. gastric haemorrhage and/or ulceration with NSAIDs; constipation is a significant side effect of the milder opiates e.g. codeine and dihydrocodeine, while the stronger, prescription opiate analgesics, e.g. tramadol, effect cognition and self-awareness, in addition to gastrointestinal side effects.

Current therapy for the management of moderate to severe pain is sub-optimal. The strongest analgesics e.g. pethidine, fentanyl, morphine and diamorphine whilst being adequate analgesics, also have significant well known side effects that often limit use e.g. tolerance over time, gastrointestinal side effects and respiratory depression. In addition, use of the strongest analgesics is strictly controlled because of their addictive properties.

Meptazinol is a mixed agonist-antagonist analgesic with specificity for the mul opioid receptor which displays both opioid and cholinergic properties and its chemical structure is defined by formula (I) below:

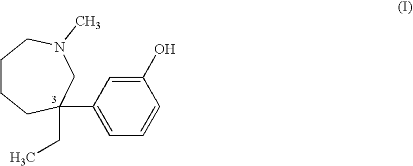

Preparation of meptazinol hydrobromide salt was referred to in U.S. Pat. No. 3,729,465 and preparation of the free base form of meptazinol was referred to in U.S. Pat. No. 4,197,241, both of which are incorporated by reference.

Meptazinol's cholinergic properties are thought to contribute to its anti-nociceptive effects and to minimize the usual range of opioid side effects. Meptazinol has been shown to have a negligible clinical dependency liability from both formal clinical investigation and the lack of reported instances of street use/abuse. The lack of addictive potential for meptazinol was first reported in 1987 by the internationally renowned investigator, Dr. Don Jasinski (Lexington, Ky.). This property distinguishes meptazinol from many other strong analgesics such as fentanyl (e.g. Duragesic), pentazocine, oxycodone (e.g. Oxycontin, Percocet), and morphine which are all classified as “Controlled Drugs” with consequent prescription/ dispensation restrictions.

Meptazinol also has many clinical advantages over the more conventional opioid analgesics which include causing minimal respiratory depression, causing minimal sedation and lacking a constipating effect.

Causing minimal respiratory depression makes meptazinol a favored obstetric analgesic to avoid infant respiratory distress. Other analgesics given during labor such as pethidine and diacetylmorphine can cause significant infant respiratory depression giving rise to the so-called grey baby syndrome often necessitating the use of a narcotic antagonist such as naloxone to reverse this effect.

Causing minimal sedation is advantageous in treating chronic pain conditions and assists a patient in conducting a normal daily life. The sedation associated with other analgesics frequently induces lethargy and a dramatic reduction in the quality of life—with the patients entering a near twilight world.

Lacking a constipating effect is an important property in treating chronic pain. The constipation commonly associated with the other strong analgesics can be a most distressing condition especially for the older patient. For this group of patients, frequently the target population for strong analgesics, the lack of a constipating effect for meptazinol represents an important advantage over other strong analgesics such as pethidine.

Additionally, age is unlikely to affect the clearance of meptazinol which is effected by a simple one-step glucuronidation process with the ensuing inactive, water-soluble conjugate being filtered at the kidney. This process of conjugative metabolic clearance is not as affected by age as some other clearance mechanisms such as direct filtration of the active entity at the kidney or oxidative metabolic clearance as required for example by pethidine.

However, despite these clinical advantages, use of meptazinol has been restricted by two major disadvantages: (1) low oral biovailability; with reported mean values lying between 4-9% as the result of extensive first pass metabolism and (2) a propensity, in common with other strong analgesics, to cause nausea and emesis. The nausea and emesis worsens bioavailability due to physical drug loss by vomiting. Furthermore, since meptazinol is known to inhibit gastric emptying and effectively traps part of the orally dosed drug in the stomach, greater quantities of meptazinol may be lost through such emesis.

All these factors lead to highly variable plasma drug levels of meptazinol after oral dosing and consequently a variable patient response. Such is the demand for immediate relief from moderate to severe pain that a patient may be unwilling to continue treatment with meptazinol until an optimal dosage is discovered for their personal use. This frustration, in attaining optimal dosage levels for each individual patient, can lead to compliance problems and ineffective medication and pain relief. The compliance issue is further exacerbated by the need for frequent oral administration of meptazinol, typically 4-6 times per day as a result of its short plasma half-life (1.5-2.0 hours).

Transdermal delivery of strong analgesics in recent years has proven to be a useful alternative to injectable delivery as a means of overcoming many of the problems associated with their oral administration. Modulating the sharp rise in plasma drug levels, usually seen after oral dosing, may serve to minimize the emesis associated with the comparatively high Cₘₐₓ values resulting from rapid absorption. In the specific case of meptazinol, avoidance of emesis becomes more important to minimize loss of drug trapped in the stomach by its inhibitory effects on gastric emptying.

Transdermal delivery also provides a means of avoiding the first pass metabolism through the liver which in the case of meptazinol removes up to 98.1% of an oral dose. Such a high first pass elimination of the drug inevitably leads to large inter and intra subject variability in achieved plasma drug concentrations. For example, in one publication (Norbury H. M, Franklin, R. A, Graham, D. F., Eur. J. Clin. Pharm., vol. 25, pgs 77-80, (1983)) oral bioavailablity varied from 1.89% to 18.5%, almost a ten-fold range.

Meptazinol is inherently not a potent drug when administered orally, requiring 200 mg dosages every four to six hours. Even when the poor bioavailability of meptazinol is factored in, the average daily required dose for an effective dose would be 50 -100 mg which approximates to a flux rate of ˜83-166 μg/cm²/h from a 25 cm² transdermal patch. Such inherently high flux rates are not usually seen with other transdermal products and so this represents a significant technical challenge.

Examples of transdermal delivery systems which generically refer to opioid analgesics including meptazinol have been referred to in the art, e.g. Oshlack et al. (U.S. Pat. No. 6,716,449); Simon (U.S. Patent Application Publication 2004-0024006); Klose et al. (U.S. Patent Application Publication 2004-0028625); Cassell (U.S. Patent Application Publication 2006-0029654); Shevchuk et al. U.S. Patent Application Publication 2004-033253) and Schlagheck (U.S. Patent Application Publication 2006-240128).

However, none of these references recognized the problem high flux rate which is uniquely associated with meptazinol and were directed toward solving the problem of delivering other types of opioid drugs (Oschlack—morphine/hydromorphone, naltrexone, oxycodone/hydrocodone; Simon—nalmefene; Klose—fentanyl; Cassell—lidocaine; Shevchuk—naltrexone, fentanyl, oxycodone+acyl opioid antagonist; Schlagheck—opioid—N-methyl-D-aspartate antagonist). There is no evidence in that any of these references solved the problem of delivering meptazinol at the necessary high flux rates or any discussion as to how this problem could be solved.

Therefore, a need still exists in the art for a transdermal delivery system for a non-addictive mixed agonist-antagonist analgesic such as meptazinol to achieve a sufficiently high flux rate to deliver a pharmacologically effective amount of the drug to treat pain or provide analgesic relief.

Citation or identification of any document in this application is not an admission that such document is available as prior art to the present invention.

### SUMMARY OF THE INVENTION

Surprisingly, the applicants have found that some or all of the disadvantages in the art with respect to the use of meptazinol can be overcome by various delivery vehicles in a transdermal device, (and most unexpectedly) with use of particular salt forms of meptazinol to provide sufficiently high flux rates to achieve plasma concentrations effective for analgesic relief.

Thus, it is an object of this invention to provide a delivery system which avoids first pass metabolism and delivers a pharmacologically effective amount of meptazinol for pain or to provide analgesic relief. The invention provides a viable means of avoiding the very large first pass effect seen with meptazinol after oral dosing. This invention will result in lower variability in achieved plasma concentrations, improved analgesic efficacy and better patient compliance.

Patient compliance will be further improved by the requirement for less frequent dosage due to the sustained plasma concentrations achieved from this transdermal delivery device according to the present invention.

Additionally, the relatively slower rise in plasma drug concentrations is expected to minimize the drug's emetic effects which again will contribute to minimizing variability in analgesically effective plasma drug concentrations and improving patient compliance

The terms “delivery system” and “delivery vehicle” as used herein is meant to describe a method of providing meptazinol via transdermal transportation which avoids “first pass metabolism”. First pass metabolism refers to the reduction of bioavailability of a drug, e.g. meptazinol, because of the metabolic or excretory capacity of the liver which is a common problem associated with oral administration. Transdermal delivery is distinct from parenteral or delivery by injection in that the latter bypasses the stratum corneum, epidermal and dermal layers of the skin and delivers the active agent directly to the subcutaneous layer. Transdermal delivery as used herein is meant to describe a process wherein an active agent, e.g. meptazinol or a derivative or precursor thereof, contacts and passes through or permeates through one or more of the stratum corneum, epidermal and dermal layers of the skin. This passing through or permeation through can be accomplished by means such as, but not limited to:
(1) transcellular penetration (across the cells);
(2) intercellular penetration (between the cells); or
(3) transappendageal penetration (via hair follicles, sweat and sebum glands, and pilosebaceous apparatus)
(4) by predisposing the stratum corneum to allow the passage of drug such as the use of thermal ablation technologies.
(5) exploiting natural transport mechanism in the skin such as that for phosphorylated vitamin E
(6) facilitated passage through the uppermost layers of the skin using microneedles

The invention disclosed herein is meant to encompass all pharmaceutically acceptable salts of meptazinol (including those of the weakly acidic phenolic function as well as those of the weakly basic azepine nitrogen). Furthermore, it encompasses various other meptazinol precursors derived by covalent linkage to the phenolic function such as ethers esters and glycosides described later. The pharmaceutically acceptable salts (of the phenol) include, but are not limited to, metal salts such as sodium salt, potassium salt, cesium salt and the like; alkaline earth metals such as calcium salt, magnesium salt and the like; organic amine salts such as triethylamine guanidine & N-substituted guanidine salts, acetamidine & N-substituted acetamidine salts, pyridine salt, picoline salt, ethanolamine salt, triethanolamine salt, dicyclohexylamine salt, N,N′-dibenzylethylenediamine salt and the like. Pharmaceutically acceptable salts (of the azepine) include, but are not limited to inorganic acid salts such as hydrochloride, hydrobromide, sulfate, phosphate and the like; organic acid salts such as trifluoroacetate, maleate, and the like; sulfonates such as methanesulfonate, ethanesulphonate, benzenesulfonate, p-toluenesulfonate, camphor sulphonate and naphthalenesulphonate, and the like; amino acid salts such as alaninate, asparginate, glutamate and the like.

Meptazinol is a chiral molecule containing one stereogenic center at the C-3 position of the azepine and can therefore exist as two enantiomeric forms (R and S stereoisomers).

Reference to meptazinol for the purposes of this invention encompasses each enantiomer and mixtures thereof including a racemic mixture (racemate) of the enantiomers unless otherwise indicated.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right and hereby disclose a disclaimer of any previously described product, method of making the product or process of using the product.

These and other embodiments are disclosed or are apparent from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the comparative permeation of meptazinol free base and a number of its salts through human skin.

FIG. 2 shows the skin flux of the various salts of meptazinol though human skin.

FIG. 3 shows an example of a meptazinol containing transdermal patch

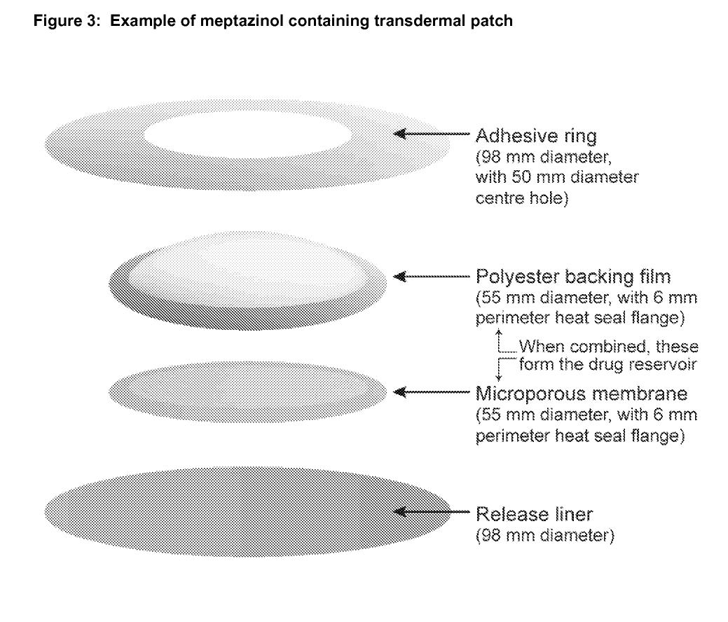

FIG. 4 shows the plasma drug concentration time profile after repeated patch application to a minipig.

### DETAILED DESCRIPTION

The present invention is directed to a delivery system which delivers a pharmacologically effective amount of meptazinol for pain or to provide analgesic relief. Examples of alternative delivery systems, include but are not limited to those means which enable delivery of meptazinol or salt form thereof via parenteral injection, pulmonary absorption, topical application, sublingual administration and rectal administration (e.g. suppositories). Parenteral injections include delivery via intravenous injection, subcutaneous injection, intramuscular injection, intraarterial injection and intrathecal injection. Pulmonary absorption includes the use of inhalants and aerosols. Topical administration includes administration via: (1) mucous membranes which includes but is not limited to mucous membranes of the conjunctiva, nasopharnyx, oropharynx, vagina, colon, urethra and urinary bladder; (2) the skin (which includes topical or transdermal delivery); and (3) the eye.

In one embodiment of the present invention, the delivery vehicle is for topical administration to the skin and includes but is not limited to a transdermal device, a gel, a cream, a lotion or an ointment which delivers a pharmacologically effective amount of meptazinol for pain or to provide analgesic relief.

In another embodiment of the invention the delivery vehicle is a transdermal device. The transdermal device is intended to deliver the pharmacologically effective amount of meptazinol either in a manner which: (1) controls the rate of drug delivery to the skin or (2) allows the skin to control the rate of drug absorption.

The transdermal device for the transdermal delivery of an effective amount of meptazinol to provide analgesic relief to a mammal or patient in need thereof comprises of:
(i) a backing layer;
(ii) a reservoir layer or compartment;
(iii) a controlling membrane or non controlling microporous membrane; and
(iv) an adhesive film which is optionally applied as a perimeter ring or as a geometric pattern or combination thereof;
(v) a release liner; and

wherein the reservoir layer or compartment contains a composition comprising
(a) a salt form of meptazinol or salt of a meptazinol precursor in an amount which results in delivery of an effective amount of meptazinol when added into the device and said device is applied to the skin; and
(b) a pharmaceutically effective carrier.

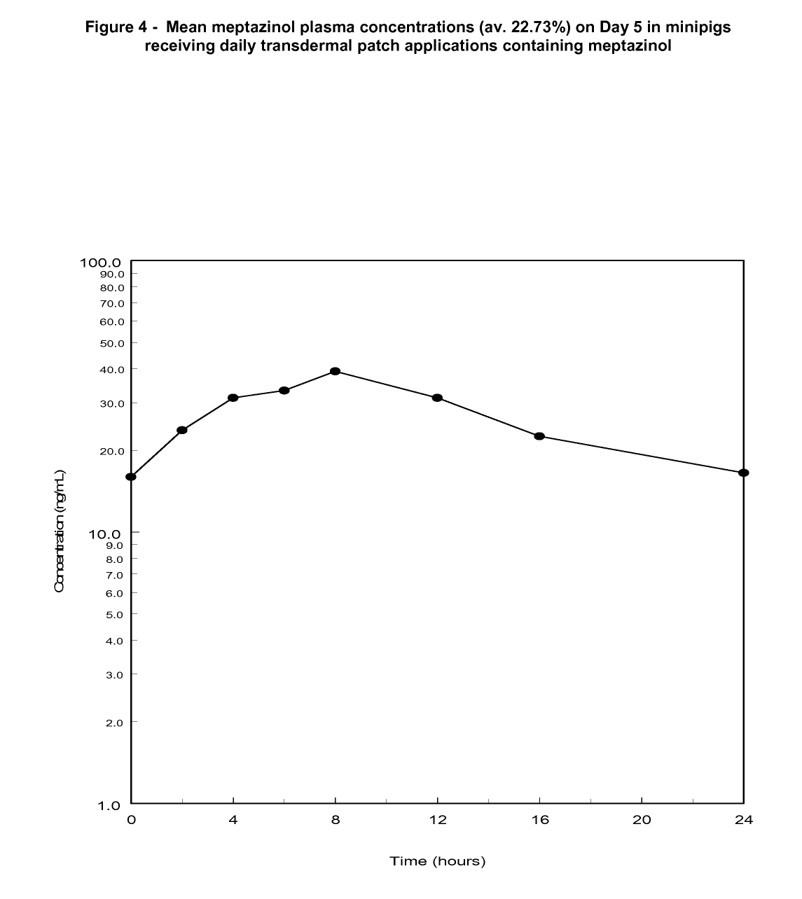

An example of the perimeter ring or geometric pattern is shown in FIG. 4, i.e. the adhesive film does not cover the entire surface area of the controlling membrane; the adhesive film is applied to enable contact with the skin while also allowing for the controlling membrane or non controlling microporous membrane to also contact the skin.

In one embodiment of the invention, the reservoir layer is a compartment and is formed from the control membrane or non controlling microporous membrane and the backing layer.

The backing layer, reservoir layer, control membrane, adhesive and release liner can be formed using conventional teachings in the art such as those referred to in U.S. Pat. No. 6,818,226 (Dermal penetration enhancers and drug delivery systems involving same); U.S. Pat. No. 6,791,003 (Dual adhesive transdermal drug delivery system); U.S. Pat. No. 6,787,149 (Topical application of opioid analgesic drugs such as morphine); U.S. Pat. No. 6,716,449 (Controlled release compositions containing opioid agonist and antagonist); U.S. Pat. No. 5,858,393 (Transdermal formulation); U.S. Pat. No. 5,612,382 (Composition for percutaneous absorption of pharmaceutically active ingredients); U.S. Pat. No. 5,464,387 (Transdermal delivery device); U.S. Pat. No. 5,023,085 (Transdermal flux enhancers in combination with iontophoresis in topical administration of pharmaceuticals; U.S. Pat. No. 4,891,377 (Trandermal delivery of the narcotic analgesics etorphine and analogs); U.S. Pat. No. 4,654,209 (Preparation of percutaneous administration), each of which is incorporated by reference.

The transdermal device of the invention is able to provide long lasting relief and is an improvement from the prior art which require 4-6 dosages per day. In one embodiment of the transdermal device is able to provide up to about 8 hours of analgesic relief, in another embodiment of the invention, the transdermal device is able to provide about 8 to about 24 hours of relief, and in a further embodiment of the invention, the transdermal device is able to provide from about 24 hours of relief to about 168 hours of relief.

Another embodiment of the transdermal device may constitute a so-called “drug in adhesive” or matrix patch in which there is no reservoir layer but instead the drug is intimately distributed in an appropriate pressure sensitive adhesive such as but not limited to the DURO-TAK polyacrylates.

In yet another embodiment of the invention, the transdermal device comprises an array of microfabricated microneedles wherein the length of the microneedle(s) is long enough to penetrate the stratum corneum (outer 10-15 μm of the skin) and yet short enough so as not to stimulate the nerves deeper into the skin. Henry et al., “Microfabricated Microneedles: A Novel Approach to Transdermal Drug Delivery”, J. Pharm. Sci., vol. 87: 922-925 (1998). The meptazinol containing composition is stored in the hollowed out section of the microneedle.

In a further embodiment of the invention the transdermal device consists of a disposable patch with an array of metallic filaments and a separate battery-operated electrical activator. A momentary pulse of current applied to the filaments through the activator creates numerous microchannels through the stratum corneum allowing the drug to subsequently permeate in a continuous manner.

A further embodiment exploits a natural transport mechanism in the skin to carry drugs across without disrupting the skin surface. This is based on the observation that phosphorylated vitamin E penetrates skin almost ten times faster than vitamin E itself. Microencapsulation of the drug within a shell of phosphorylated vitamin E, creating nanospheres, then enables the drug to be efficiently carried across the skin. Continuous delivery over an extended period is then achievable.

In another embodiment of the invention, transdermal drug delivery is enhanced by iontophoresis, magnetophoresis, or sonophoresis. Iontophoresis involves the delivery of charged chemical compounds across the skin membrane using an applied electrical field. see e.g. “Pharmaceutical Dosage Forms and Drug Delivery Systems—Chapter 10—Transdermal Drug Delivery Systems, Ointments, Creams, Lotions and Other Preparations”, ed. by Ansel et al., Williams & Wilkins, page 360, (1995). Magnetophoresis involves the use of a magnetic field to enhance drug delivery to the skin. see e.g. Murthy et al., “Physical and Chemical Permeation Enhancers in Transdermal Delivery of Terbutaline Sulphate”, AAPS Pharm Sci Tech. 2001; 2(1). Sonophoresis is the use of high-frequency ultrasound which serves to compromise the integrity of the stratum corneum layer and improve permeability of compounds through the skin.

Given the low solubility of the free base form of meptazinol free base (0.17 mg/mL in aqueous solution), it may be advantageous to derivatize the meptazinol to form a precursor compound (or a salt thereof) which will degrade into meptazinol when traversing the layer(s) of the skin. Therefore, another embodiment of the invention is the delivery of meptazinol transdermally which is achieved by a transdermal device which contains a precursor of meptazinol which includes but is not limited to meptazinol esters, glycosides, salts of meptazinol or mixtures thereof. Precursors of meptazinol are compounds which undergo a transformation in vivo to produce meptanizol (e.g. cleavage of an ester bond, glycolysis, formation of the free base from the salt). Meptazinol esters, ethers and glycosides of the invention are compounds of the formula (II): 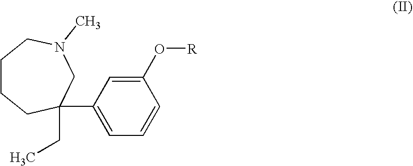
wherein R is an acyl group, a mono-, oligo- or poly-saccharide, or salts of mono-, oligo- or poly-saccharides. (Oligosaccharide for the purpose of this invention indicates a saccharide comprised of 2-10 monosaccharide units which are covalently bonded together).

When R forms an ester, one embodiment of the invention is where R is a —C(═O)—C₁-C₁₂-alkyl; yet another embodiment is where R is —C(═O)—C₁-C₁₂-alkyl-NR₁R₂ wherein R₁ and R₂ are independently hydrogen or C₁-C₄ alkyl; yet another embodiment is where R is C(═O)—C₁-C₁₂-alkylCO₂R₃ wherein R₃ is hydrogen, C₁-C₄ alkyl or is a cation.

In a further embodiment of the invention, R is a —C(═O)—C₁-C₄-alkyl; yet another embodiment is where R is —C(═O)—C₁-C₄-alkyl-NR₁R₂ wherein R₁ and R₂ are independently hydrogen or C₁-C₄ alkyl; yet another embodiment is where R is C(═O)—C₁-C₄-alkylCO₂R₃ wherein R₃ is hydrogen, C₁-C₄ alkyl or is a cation.

When R forms an ether, one embodiment of the invention is where R is a substituted or unsubstituted C₁-C₁₂-alkyl or substituted or unsubstituted aryl. In another embodiment of when R is an ether, R is a substituted or unsubstituted C₁-C₄-alkyl or substituted or unsubstituted phenyl. In both embodiments, the substituents are selected from the group consisted of halogen, C₁-C₄-alkyl, and C₁-C₄-alkoxy.

When R is a monosaccharide, one embodiment of the invention is where R is selected from the group consisting of erythrosyl, threosyl, ribosyl, arabinosyl, xylosyl, lyxosyl, allosyl, altrosyl, glucosyl, glucosylamino, mannosyl, gulosyl, idosyl, galactosyl, galactosylamino, talosyl and salts thereof, another embodiment is where R is glucosyl, glucosylamino, galactosyl or galactosylamino and salts thereof, and yet another embodiment of the invention is where R is glucosyl and salts thereof.

When R is an oligosaccharide, one embodiment of the invention is where R is selected from the group consisting of lactose, sucrose, trehalose, Lewis a trisaccharide, 3′-O-sulfonato Lewis a, Lewis b tetrasaccharide, Lewis x trisaccharide, Sialyl Lewis x, 3′-O-sulfonato Lewis x, Lewis y tetrasaccharide and salts thereof.

When R is a polysaccharide, one embodiment of the invention is where R is selected from the group consisting of chitin, chitosan, cyclodextrin, dextran and pullulan; another embodiment of the invention is where the cyclodextrin is α-, β- or γ-cyclodextrin; yet another embodiment of the invention is where the cyclodextrin is β-cyclodextrin, dimethyl-β-cyclodextrin or hydroxypropyl-β-cyclodextrin.

As the cyclodextrin have a cavity which can accommodate the inclusion of a compound such as meptazinol, another embodiment of the invention is where the cyclodextrins described in R above can also be added to meptazinol to form an inclusion complex rather than being linked covalently.

In another embodiment of the invention, the meptazinol precusor is a salt and R is hydrogen but absent, whereby the oxygen is negatively charged; one embodiment of the invention is where the salt form is selected from the group consisting of sodium, potassium, caesium, calcium, magnesium, guanidine & N-substituted guanidine salts and acetamidine & N-substituted acetamidine salts triethylamine, pyridine, picoline, ethanolamine, triethanolamine, dicyclohexylamine, N,N′-dibenzylethylenediamine. Another embodiment of the invention is when R is hydrogen—or one of the aforementioned substituents—and the azepine nitrogen is positively charged and linked with hydrochloride, hydrobromide, sulfate, phosphate, formate, acetate, trifluoroacetate, maleate, tartrate, methanesulfonate, ethanesulphonate, benzenesulfonate, p-toluenesulfonate, naphthalene sulphonate. camphor sulfonate, arginate, alaninate, asparginate, glutamate and mixtures thereof.

Surprisingly, and contrary to prior notions that lower melting points (mp) are normally associated with improved skin permeability, the azepine salts of meptazinol do not show such a relationship. For example, meptazinol hydrochloride (mp 184° C.) was a much better permeant than the maleate (mp 102-104° C.). The hydrochloride also displayed a higher flux rate than the camsylate (mp of 46-48° C.).

Furthermore, and again in contrast to prior notions in the art, additional unexpected results occurred when using salts of meptazinol for transdermal delivery. Usually the free base is the preferred form of a drug for transdermal delivery due to its greater lipophilicty. For example, the skin flux of fentanyl free base is up to five times faster than the salt form. However, for meptazinol, the free base shows unexpectedly poor flux in comparison to the various salt forms. For example, meptazinol hydrochloride has a substantially greater flux than the free base. Previous reports in the scientific literature have suggested that ion pairs, i.e. salts, may improve transdermal flux by virtue of beneficially enhancing the physicochemical characteristics of the molecule. Such strategies have often employed lipophilic counter ions. Surprisingly, in the case of meptazinol, the use of more lipophilic counter ions such as the camsylate and tosylate were less effective in improving flux than the use of salts of stronger acid such as trifluoroacetic acid or hydrochloric acid.

A further factor improving the overall rate of skin flux was the unexpected radial or lateral diffusion of the meptazinol; this is advantageous in that higher skin fluxes can allow for smaller diameters of patch sizes

In another embodiment of the invention, an additional analgesic can be added to the transdermal device. Examples of analgesics include but are not limited to ethanol, non-steroidal anti-inflammatory drugs (NSAIDs) and other compounds with anagelsic properties such as but not limited to amitriptyline and carbamazepine.

In another embodiment of the invention, the only analgesic present in the composition in the reservoir layer is a salt of meptazinol or a salt of a meptazinol precursor.

In another embodiment of the invention, the pharmaceutically effective carrier includes but is not limited to a solvent such as alcohol, isopropylmyristate, glycerol monooleate or a diol such as propylene glycol, or the like. The delivery of the meptazinol or meptazinol precursor (or salts thereof) is enhanced by the use of a permeation enhancer which may also be included in the pharmaceutically effective carrier. In one embodiment of the invention, suitable permeation enhancers include but are not limited to polyunsaturated fatty acids (PUFA) such as arachidonic acid, lauric acid, α-linolenic acid, linoleic acid and oleic acid; dimethylisosorbide; azones; cyclopentadecalactone; alkyl-2-(N,N-disubstituted amino)-alkanoate ester (NexAct); 2-(n-nonyl)-1,3-dioxaolane (SEPA); cod-liver oil; essential oils, glycerol monoethers derived from saturated fatty alcohols; D-limonene; menthol and menthol ethyl ether; N-methyl-2-pyrrolidone (NMP); phospholipids; squalene; terpenes; and alcohols such as methanol, ethanol, propanol and butanol. see e.g. Pharmaceutical Skin Penetration Enhancement, ed. Walters et al., Marcel Dekker, Inc., (1993); Williams et al., “Penetration Enhancers”, Adv. Drug Deliv. Rev., vol. 56, pgs 603-618, (2004).

Alternatively, in another embodiment of the invention transdermal drug delivery may be effected using various topically applied ointments, creams, gels or lotions. Typically these may comprise oil-in-water emulsions or water-in-oil emulsions incorporating meptazinol or meptazinol precursor in one of the preferred vehicles. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

In another embodiment of the invention, the transdermal drug delivery is made concomitant with oral administration of a composition containing an analgesic agent.

In another embodiment of the invention, the solubility (as measured in aqueous solution) of the meptazinol or meptazinol precursor or salt thereof is about 30 mg/mL to about 500 mg/mL; in yet another embodiment of the invention, the solubility is about 50 mg/mL to about 400 mg/mL; and in a still further embodiment of the invention, the solubility is about 75 mg/mL to about 300 mg/mL.

Skin flux can be determined by multiplying the permeability coefficient (kₚ in cm/h) of meptazinol by the aqueous solubility of meptazinol. The aqueous solubility of meptazinol (free base) is 0.17 mg/mL and the permeability coefficient of meptazinol can be calculated using the empirical formula:
log kₚ=−2.7+0.71 log P−0.0061 MW (MW for meptazinol is 233.35)
This results in an estimated skin flux of just 5.6 μg/cm²/h for meptazinol which is roughly 15-30× lower than the flux rate necessary to achieve analgesic effects through transdermal delivery.

In another embodiment of the invention, the skin flux for the delivery of the meptazinol or meptazinol precursor or salt thereof is about 20 to about 1000 μg/cm²/h; in yet another embodiment of the invention, the skin flux for the delivery of the meptazinol or meptazinol precursor is about 50 to about 500 μg/cm²/h; in a further embodiment of the invention is about 125 to about 250 μg/cm²/h and in a still further embodiment of the invention is about 160 to about 200 μg/cm²/h.

In another embodiment of the invention the pH of the environment into which the meptazinol or meptazinol precursor or salt thereof is released is pH 2.0 to about pH 4.0; in another embodiment about about pH 4.0 to about pH 7.0; in yet another embodiment, the pH is about 4.0 to about 6.0; and in a further embodiment, the pH is about 4.0 to about 5.0.

Optionally, additional skin care ingredients may be combined with meptazinol or the meptazinol precursors or salt thereof for their art recognized effects, these include abrasives, absorbents, adhesives, antiacne agents, anticaking agents, anticareis agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, depilating agents, drug astringents, emollients, emulsion stabilizers, epilating agents, exfoliants, external analgesics, film formers, flavoring agents, fragrance ingredients, gelling agents, humectants, lytic agents, occlusives opacifying agents, oxidizing agents, pesticides, pH adjusters, plasticizers, preservatives, propellants, reducing agents, skin-bleaching agents, skin-conditioning agents, skin protectants, slip modifiers, solvents, sunscreen agents, surface modifiers, surfactants (including cleansing agents, emulsifying agents, foam boosters, hydrotopes, solubilizing agents, suspending agents), suspending agents (non-surfactant), ultraviolet light absorbers, viscosity controlling agents, viscosity decreasing agents, viscosity increasing agents (aqueous), viscosity increasing agents (non-aqueous) and mixtures thereof.

These additional skin care ingredients include but are not limited to those described in The International Cosmetic Ingredient Dictionary and Handbook, 9^{th} Edition (2002); Remington—The Science and Practice of Pharmacy, 21^{st} Edition (2005), Goodman & Gilman's The Pharmacological Basis of Therapeutics, 11^{th} Edition (2005) and Ansel's Parmaceutical Dosage Forms and Drug Delivery Systems (8^{th} Edition), edited by Allen et al., Lippincott Williams & Wilkins, (2005).

Controlling the release rate of the meptazinol composition and avoiding compromising the stickiness of the perimeter ring of the adhesive layer of the transdermal device is also desired. As such another embodiment of the invention is to add a gelling agent to the meptazinol composition. Suitable gelling agents include but are not limited to Klucel (hydroxypropyl cellulose) and Carbopol 980. Such gelling agents, by virtue of the viscosity they provide , will control and restrict the rate of vehicle delivery through the microporous membrane. Typically this may be of the order of 1.5-3.0 uL/cm²/h. By such control of the rate of delivery of drug and vehicle to the skin surface this may serve to limit any unwanted skin irritancy.

One of the side effects of transdermal delivery of an active agent is indeed the occurrence of skin irritation. While not wishing to be bound by theory, meptazinol may cause skin irritation due to the formation of oxidized or degradative products (e.g. meptazinol 1,4 quinone, meptazinol dimers). Furthermore, there is a possibility of dimerization of this quinone could be responsible for the pronounced yellow discoloration of the gel observed on standing. It has been found that this discoloration can be completely eliminated by the inclusion of the antioxidant butylated hydroxy toluene (BHT—0.02-0.05%). Ascorbic acid (0.05%) also afforded some reduction in this yellow discoloration, but other anti-oxidants such as butylated hydroxy anisole, alpha tocopherol and pyrogallol did not. Lesser amounts of BHT and ascorbic acid may also be suitable for the meptazinol compositions of the invention.

As such, another embodiment of the invention is a meptazinol containing composition for transdermal delivery which is free from skin irritation which further comprises an antioxidant selected from the group consisting of BHT, ascorbic acid and mixtures thereof.

Another factor which could induce skin irritation could, in part, be an inappropriate pH especially lower pH's. This can be raised by the use of a pharmaceutically acceptable basifying agent such as, but not exclusively restricted to, diethanolamine, disopropanolamine or tromethamine(TRIS).

In another embodiment of the invention, use of the transdermal device described hereinabove can be used to provide analgesic effects to treat systemic or localized pain to a patient in need thereof.

Yet another embodiment of the invention is a method of delivering meptazinol which avoids first pass metabolism which comprises of transdermal delivery. In another embodiment of this invention the method of delivery is non-oral and/or non-parenteral.

Other advantages and characteristics of the invention will become apparent on reading the following description, given by way of non-limiting examples.

### EXAMPLES

### Example 1

### Improved Skin Flux by Using Meptazinol HCl Salt

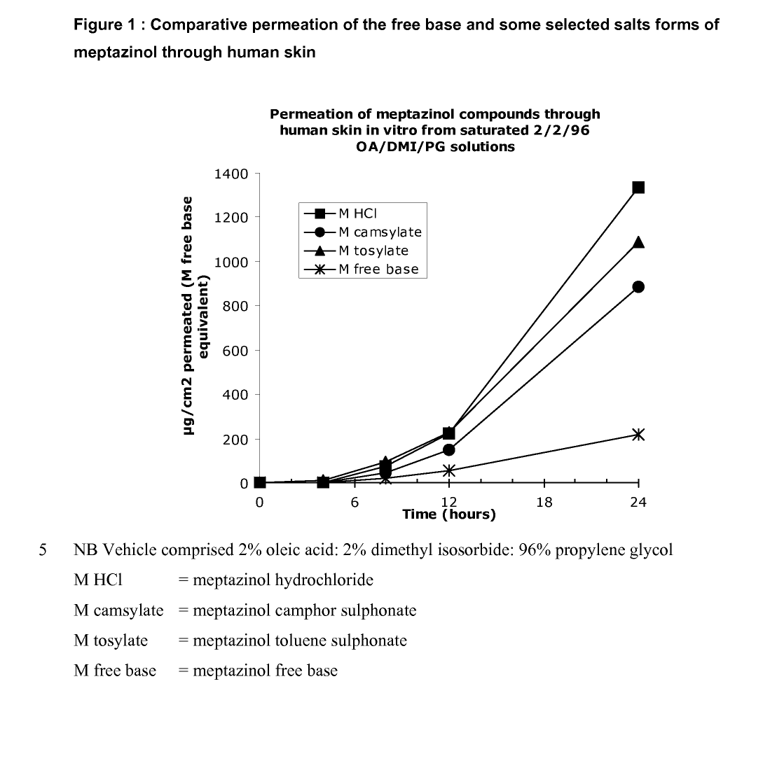

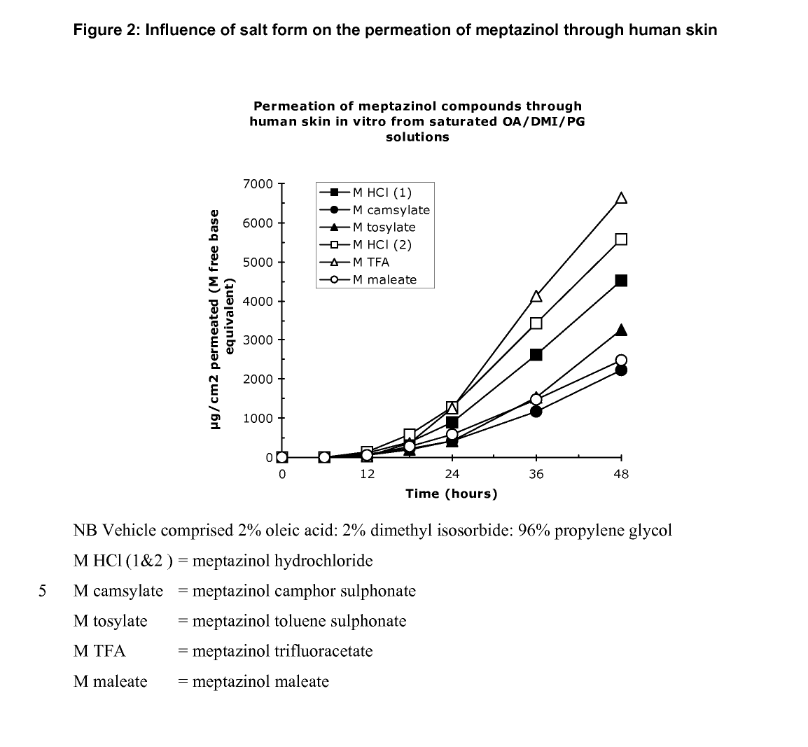

Using human skin in a conventional Franz cell in vitro apparatus the transdermal permeation of meptazinol was measured by assaying the amount of drug in the receptor fluid beneath the skin sample at various times after application to the skin. FIG. 1 shows that a salt of meptazinol is surprisingly more permeable than the free base form of meptazinol. FIG. 2 shows that surprisingly meptazinol salts formed from a stronger acid, such as the hydrochloride and trifluoroaceate salt are more rapidly absorbed than are those of weaker organic acids such as the camsylate, tosylate or maleate.

The data presented in Table 1 below show that under the test conditions cited above, the mean flux for the various salts tested were suitable for producing concentrations of meptazinol sufficient to produce a long-lasting effect when administered to a patient in need thereof.

TABLE 1
Intersubject variability in flux rates for meptazinol
salts through human skin

| Applied | | Donor | Flux | |
|---|---|---|---|---|
| compound | Cell ID | number | ug/cm²/h | Mean flux ± sd |
| M HCl (1) | 1 | 281 | 197.1 | 173.3 ± 35.2 |
| M HCl (1) | 2 | 282 | 194.0 | |
| M HCl (1) | 3 | 284 | 162.5 | |
| M HCl (1) | 4 | 287 | 135.7 | |
| M HCl (1) | 5 | 289 | 218.0 | |
| M HCl (1) | 6 | 295 | 132.6 | |
| M camsylate | 7 | 281 | 78.1 | 79.2 ± 71.2 |
| M camsylate | 8 | 282 | 110.9 | |
| M camsylate | 9 | 284 | 27.3 | |
| M camsylate | 10 | 287 | 45.1 | |
| M camsylate | 11 | 289 | 204.2 | |
| M camsylate | 12 | 295 | 9.5 | |
| M tosylate | 13 | 281 | 158.8 | 135.9 ± 82.5 |
| M tosylate | 14 | 282 | 165.0 | |
| M tosylate | 15 | 284 | 90.9 | |
| M tosylate | 16 | 287 | 39.6 | |
| M tosylate | 17 | 289 | 273.7 | |
| M tosylate | 18 | 295 | 87.4 | |
| M HCl (2) | 19 | 292 | 66.5 | 179.0 ± 75.1 |
| M HCl (2) | 20 | 288 | 222.6 | |
| M HCl (2) | 21 | 282 | 213.4 | |
| M HCl (2) | 22 | 281 | 213.4 | |
| M TFA | 23 | 292 | 29.1 | 224.4 ± 167.6 |
| M TFA | 24 | 288 | 422.2 | |
| M TFA | 25 | 282 | 163.8 | |
| M TFA | 26 | 281 | 282.3 | |
| M maleate | 27 | 292 | 41.3 | 78.0 ± 24.7 |
| M maleate | 28 | 288 | 91.3 | |
| M maleate | 29 | 282 | 85.3 | |
| M maleate | 30 | 281 | 93.9 | |

NB Vehicle comprised 2% oleic acid: 2% dimethyl isosorbide: 96% propylene glycol The data in Table 2 was obtained using the same in vitro Franz cell technique verifies that there was surprisingly no correlation between lower melting points and higher solubilities with overall skin flux rates. For example, based on prior notions in the art, meptazinol hydrochloride which has a higher melting point than meptazinol free base, would have been expected to have a worse skin flux rate but instead is several times better than the meptazinol free base. Likewise, meptazinol hydrochloride which is the salt of a stronger acid, has both lower solubility and higher melting point than meptazinol camsylate, tosylate or maleate which is the salt of a weaker acid, and yet still have an unexpectedly better skin flux rate.

TABLE 2
Saturated solubilities (& melting points) of meptazinol free
base and selected salts in a potential delivery vehicle comprising
2% oleic acid: 2% dimethyl isosorbide: 96% propylene glycol

| Compound | Solubility at 32° C. (mg/ml) | MP (° C.) |
|---|---|---|
| Meptazinol hydrochloride | 78* | 184-186 |
| Meptazinol camsylate | ˜250** | 46-48 |
| Meptazinol tosylate | ˜140** | 40-42 |
| Meptazinol trifluoroacetate | 170* | 112-114 |
| Meptazinol maleate | 105* | 102-104 |
| Meptazinol free base | ˜20** | 128-133 |

*Measured by HPLC
**Estimated from visual assessment only

### Example 2

### Meptazinol Composition

Numerous studies using skin collected from cosmetic surgical procedures in women (usually ‘tummy tucks’) were conducted in order to establish and refine the composition of the transdermal gel. These studies culminated in the selection of a 3:2:95 weight ration of (OA:DI:PG) vehicle (OA—oleic acid; DI—dimethyl isosorbide; PG—propylene glycol)

A meptazinol gel composition for use with a transdermal patch was prepared by mixing together the following ingredients (all % by weight):

| | |
|---|---|
| 83.296% | Propylene glycol (PG) - EP (BASF and Inovene) |
| 2.63% | Oleic acid (GA) - Super Refined Oleic Acid NF/EP (Croda) |
| 1.754% | Dimethyl isosorbide (DI) - Arlasolve ™ (Uniqema) |
| 0.8% | Hydroxypropyl cellulose - Klucel HF grade NF/EP |
| | (Hercules) |
| 0.02% | Butylated hydroxytoluene (BHT) - EP grade (Fluka) |
| 11.5% | Meptazinol HCl BP grade (Kern Pharma) |

Note:
the weight ratio of (OA:DI:PG) in isolation is 3:2:95.

### Example 4

### Meptazinol Containing Transdermal Patch

FIG. 4 shows an example of a meptazinol containing transdermal patch which was prepared in accordance with the invention.

ScotchPak 9742 fluoropolymer with a thickness of 4.6 mil and 98 mm diameter was used to form the release liner (1). DSM Solupor 10PO5A which has a 55 mm diameter with a 6 mm perimeter heat seal flange was used to form the microporous membrane (2). Amcor C FILM (Amcor Flexibles Inc.) with a 6 mil thickness, 55 mm diameter with a 6 mm perimeter heat seal flange (or alternatively, 54 mm diameter with a 5 mm heat seal flange) was used to form the backing film (3). Dow Corning BIO PSA 7-4302 adhesive mixed with 2.5% of Dow Corning 200 fluid (tack enhancer) was used to form the adhesive ring (4) which has a diameter of 98 mm with a 50 mm diameter center hole (coating weight of the adhesive ring (4) was 85 g/m²).

The drug reservoir is formed by the combination of the microporous membrane (2) and the backing film (3) and has a fill volume of 100 μL/cm². The meptazinol composition contained in the drug reservoir was a 2.5 mL composition described in Example 3.

### Example 5

### In Vivo Transdermal Absorption Studies in Minipigs

Two studies were conducted in Gottingen minipigs, involving daily application of the patch of Example 4 for up to 7 days. This patch comprised a gel reservoir (100 μL/cm², nominal initial volume 2.5 mL) over a Solupor 10PO5A microporous membrane (surface area 25 cm²) This was secured to the skin via a perimeter ring of adhesive and an adhesive overlay. On a separate occasion, the pigs were given an i.v. dose of meptazinol at ˜1 mg/kg to enable bioavailability to be determined.

The results of this study showed that between 150-200 mg of drug left the patch over a 24-hour period. The pharmacokinetic profile showed a negligible lag time and time of maximum concentration (tₘₐₓ) occurring within 8 hours. Plasma levels were sustained at steady state, with a mean fluctuation index of only 2.3. The transdermal bioavailability was low, being of the order of 8% to 12%, possibly as the result of skin metabolism in the minipigs. However, no such extensive glucuronidation has been reported for other phenolic analgesics applied to human skin (Roy S D, Hou S Y, Witham S L, Flynn G L, “Transdermal delivery of narcotic analgesics: comparative metabolism and permeability of human cadaver skin and hairless mouse skin”, J Pharm Sci., vol. 83(12):1723-8 (1994)).

This surprising gain in the rate of skin flux was thought to be the result of rapid radial or lateral diffusion of the meptazinol salt when a gelling agent was added.

### Example 6

### Protocol for In Vivo Skin Flux Determination in Humans

The following protocol is to be practiced to determine the transdermal flux of a meptazinol gel formulation and to determine the systemic availability compared to an intravenous (IV) administration. In addition, the protocol also allows for an assessment of safety and local tolerance of meptazinol transdermal gel compared to an IV (intravenous) administration of meptazinol and a gel placebo.
Treatment A: Meptazinol gel and/or patch as described in Examples 3 and 4 above, will be applied and secured to the inside of the lower arm of ten volunteers and occluded for 24 hours plus a concurrent single dose of intravenous placebo (sterile 0.9% w/v sodium chloride) 0.5 mL injection infused over 30 seconds (slow IV injection). The meptazinol gel will be administered to provide for a total topical dose of 129 mg meptazinol (free base). The concentration of meptazinol hydrochloride in gel will be 100 mg/mL; 0.1 mL/cm² gel will cover a skin surface area of 13 cm². This will be contained within a stoma ring of 4 cm diam. beneath which a DSM 10PO5A microporous membrane will be placed. The ring will be occluded with a watch glass.
Treatment B: Matching gel placebo applied and secured to the inside of the lower arm and occluded for 24 hours plus a concurrent 50 mg single dose of IV meptazinol 0.5 mL injection infused over 30 seconds (slow IV injection).
   All subjects will receive both treatments over two dose periods. Subjects will be randomized with respect to the sequence of treatments will be selected to provide for adequate assessment of safety, tolerability, and pharmacokinetics.

This protocol is a randomized, two way cross over design for a treatment sequence (AB or BA). Treatments will be administered on days 1 and 4 of the study. Gel (meptazinol formulation or placebo) will be applied to the skin and left for 24 hours. IV meptazinol or the matching IV placebo will be administered as a slow bolus infusion in the arm opposite to that receiving gel. On day 1 gel will be applied to the right arm and on day 4 gel will be applied to the left arm.

A series of plasma and urine PK samples will be collected and skin will be assessed for tolerance of gel during the inpatient period of the study.

This protocol will evaluate meptazinol (and possible metabolites) plasma concentration-time profiles and pharmacokinetic parameters to include Cₘₐₓ, tₘₐₓ, AUC, t_{1/2} and bioavailability of meptazinol administered by transdermal gel relative to intravenous administration for each subject; estimation of transdermal flux; amount of meptazinol (and possible metabolites) excreted in urine; local tolerance assessed by visual inspection of skin, erythema and oedema, as well as testing the individual for itchiness, burning or other discomfort at the site of gel application supported by digital photography of application site.

Having thus described in detail various embodiments of the present invention, it is to be understood that the invention defined by the above paragraphs is not to be limited to particular details set forth in the above

What is claimed is:

1. A transdermal device for the transdermal delivery of an effective amount of meptazinol to provide analgesic relief to a mammal or patient in need thereof which comprises:
(i) a backing layer;
(ii) a reservoir layer or compartment;
(iii) a controlling membrane or non controlling microporous membrane;
(iv) an adhesive film which is optionally applied as a perimeter ring or as a geometric pattern or combination thereof;
(v) a release liner; and
wherein the reservoir layer contains a composition comprising
(a) a salt form of meptazinol or salt of a meptazinol precursor in an amount which results in delivery of an effective amount of meptazinol when added into the device and said device is applied to the skin; and
(b) a pharmaceutically effective carrier.

2. The transdermal device of claim 1, wherein the device further comprises a control membrane and an adhesive and optionally a release liner.

3. The transdermal device of claim 1, wherein the device is able to provide analgesic relief for a time period selected from the group consisting of about 8 hours of analgesic relief, and about 24 hours of relief to about 168 hours of relief.

4. The transdermal device of claim 1, which comprises a salt of a meptazinol precursor.

5. The transdermal device of claim 4, wherein the meptazinol precursor has the formula (II): 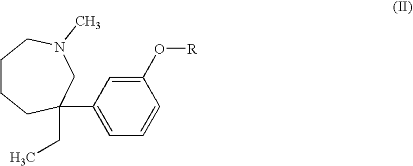
wherein,
R forms an ester, ether or glycoside with —O—; or
wherein
R is hydrogen or absent, whereby the oxygen is negatively charged, a salt of meptazinol.

6. The transdermal device of claim 5, wherein:
when R forms an ester, R is a —C(═O)—C₁-C₁₂-alkyl; —C(═O)—C₁-C₁₂-alkyl-NR₁R₂ wherein R₁ and R₂ are independently hydrogen or C₁-C₄ alkyl; or R is C(═O)—C₁-C₁₂-alkylCO₂R₃ wherein R₃ is hydrogen, C₁-C₄ alkyl or is a cation;
when R forms an ether, R is a substituted or unsubstituted C₁-C₁₂-alkyl or substituted or unsubstituted aryl;
when R forms a glycoside, R is selected from the group consisting of monosaccharide, oligosaccharide, polysaccharide, erythrosyl, threosyl, ribosyl, arabinosyl, xylosyl, lyxosyl, allosyl, altrosyl, glucosyl, glucosylamino, mannosyl, gulosyl, idosyl, galactosyl, galactosylamino, talosyl and salts thereof; another embodiment is where R is glucosyl, glucosylamino, galactosyl, galactosylamino, lactose, sucrose, trehalose, Lewis a trisaccharide, 3′-O-sulfonato Lewis a, Lewis b tetrasaccharide, Lewis x trisaccharide, Sialyl Lewis x, 3′-O-sulfonato Lewis x, Lewis y tetrasaccharide, chitin, chitosan, cyclodextrin, dextran, pullulan; α-cyclodextrin, β-cyclodextrin, γ-cyclodextrin, dimethyl-β-cyclodextrin, hydroxypropyl-β-cyclodextrin and salts thereof, or where R is an amino acid e.g. —C-alkyl NH₂.
when R is hydrogen but absent, and the oxygen is negatively charged, a salt of meptazinol wherein the salt form is selected from the group consisting of sodium, potassium, secium, calcium, magnesium, triethylamine, pyridine, picoline, ethanolamine, triethanolamine, dicyclohexylamine, N,N′-dibenzylethylenediamine.

7. The transdermal device of claim 6, wherein R is hydrogen and the nitrogen in formula (II) is positively charged and the salt form is hydrochloride, hydrobromide, sulfate, phosphate, trifluoroacetate, maleate, tartrate, methanesulfonate, ethanesulphonate, benzenesulfonate, p-toluenesulfonate, naphthalene sulphonate, camphor sulfonate, alaninate, asparginate, glutamate or mixtures thereof.

8. The transdermal device of claim 7, wherein the salt form is hydrochloride, camphor sulphonate, toluene sulfonate, trifluoroacetate, maleate or mixtures thereof.

9. The transdermal device of claim 8, wherein the salt of meptazinol has a solubility selected from the group consisting of about 30 mg/mL to about 500 mg/mL; about 50 mg/mL to about 400 mg/mL; and about 75 mg/mL to about 300 mg/mL.

10. The transdermal device of claim 8, wherein the salt of meptazinol has a skin flux selected from the group consisting of about 20 to about 1000 μg/cm²/h; about 50 to about 500 μg/cm²/h; about 125 to about 250 μg/cm²/h and about 160 to about 200 μg/cm²/h.

11. The transdermal device of claim 8, wherein the salt of meptazinol is released into an skin environment with a pH selected from the group consisting of about pH 2 to about pH about pH 4.0; about pH 4.0 to about pH 7.0; about pH 4.0 to about pH 6.0; and about pH 4.0 to about pH 5.0.

12. The transdermal device of claim 1, which comprises of a salt of meptazinol wherein the salt form is hydrochloride, trifluoroacetate or mixtures thereof, has a solubility of about 75 mg/mL to about 300 mg/mL and a skin flux of about 75 to about 250 μg/cm²/h.

13. The transdermal device of claim 10, which additionally comprises abrasives, absorbents, adhesives, antiacne agents, anticaking agents, anticareis agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, depilating agents, drug astringents, emollients, emulsion stabilizers, epilating agents, exfoliants, external analgesics, film formers, flavoring agents, fragrance ingredients, humectants, lytic agents, occlusives.opacifying agents, oxidizing agents, pesticides, pH adjusters, plasticizers, preservatives, propellants, reducing agents, skin-bleaching agents, skin-conditioning agents, skin protectants, slip modifiers, solvents, sunscreen agents, surface modifiers, surfactants (including cleansing agents, emulsifying agents, foam boosters, hydrotopes, solubilizing agents, suspending agents), suspending agents (non-surfactant), ultraviolet light absorbers, viscosity controlling agents, viscosity decreasing agents, viscosity increasing agents (aqueous), viscosity increasing agents (non-aqueous) and mixtures thereof.

14. The transdermal device of claim 12, which additionally comprises abrasives, absorbents, adhesives, antiacne agents, anticaking agents, anticareis agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, depilating agents, drug astringents, emollients, emulsion stabilizers, epilating agents, exfoliants, external analgesics, film formers, flavoring agents, fragrance ingredients, humectants, lytic agents, occlusives.opacifying agents, oxidizing agents, pesticides, pH adjusters, plasticizers, preservatives, propellants, reducing agents, skin-bleaching agents, skin-conditioning agents, skin protectants, slip modifiers, solvents, sunscreen agents, surface modifiers, surfactants (including cleansing agents, emulsifying agents, foam boosters, hydrotopes, solubilizing agents, suspending agents), suspending agents (non-surfactant), ultraviolet light absorbers, viscosity controlling agents, viscosity decreasing agents, viscosity increasing agents (aqueous), viscosity increasing agents (non-aqueous) and mixtures thereof.

15. A method of providing analgesic effect to a patient in need thereof which comprises administering the transdermal device of claim 1.

16. The method of claim 15, wherein the analgesic effect is localized.

17. The method of claim 15, wherein the analgesic effect is systemic.

18. The method of claim 15, wherein administration of the transdermal device is accompanied by iontophoresis.

19. The method of claim 15, wherein administration of the transdermal device is accompanied by magnetophoresis.

20. The method of claim 15, wherein administration of the transdermal device is accompanied by sonophoresis.

21. The method of claim 15, wherein transdermal delivery is effected by use of a lotion, cream or ointment in place of a device.

22. The method of claim 15, wherein transdermal delivery is effected using a matrix patch in which the drug is dissolved in a suitable pressure sensitive adhesive.

23. The method of claim 15, wherein administration of the transdermal device is accompanied by thermal ablation.

24. The method of claim 15, wherein administration of the transdermal device is accompanied by facilitated by a natural skin transporter of phosphorylated Vit E.

25. The method of claim 15, wherein administration of the transdermal device is accompanied by use of microneedles.

26. A method of delivering meptazinol which avoids first pass metabolism which comprises of transdermal delivery.
